(19) United States
(12) Patent Application Publication
(10) Pub. No.: US 2005/0267560 A1
(43) Pub. Date: Dec. 1, 2005

(54) Implantable bioabsorbable valve support frame

(75) Inventor: Brian L. Bates, Bloomington, IN (US)

Correspondence Address: BRINKS HOFER GILSON & LIONE, P.O. BOX 10395, CHICAGO, IL, 60610, US

(73) Assignee: Cook Incorporated

(21) Appl. No.: 11/136,039

(22) Filed: May 23, 2005

Related U.S. Application Data
(63) Continuation-in-part of application No. 09/777,091, filed on Feb. 5, 2001.
(60) Provisional application No. 60/180,002, filed on Feb. 3, 2000.
(60) Provisional application No. 60/575,230, filed on May 28, 2004.

(51) Int. Cl. A61F 2/06

(52) U.S. Cl. 623/1.1

(57) ABSTRACT

Medical devices for implantation within a body vessel comprising a frame formed at least in part from a metallic bioabsorbable material are provided. The devices can be pushed from a delivery catheter into the lumen of a duct or vessel and may include one or more barbs for anchoring purposes. A full or partial covering of fabric or other flexible material, or a bioabsorbable material, including a collagen-based material such as small intestinal submucosa (SIS), may be attached to the frame to form an occlusion device, a graft, or an implantable, intraluminal valve such as for correcting incompetent venous valves.

This application claims priority to U.S. Provisional Patent Application Ser. No. 60/575,230, filed May 28, 2004, and incorporated herein by reference in its entirety; this application is also a continuation-in-part of U.S. Utility patent application Ser. No. 09/777,091, filed Feb. 5, 2001 and incorporated herein by reference in its entirety (published as U.S. 2001/0039450 A1 on Nov. 8, 2001), which claims priority to U.S. Provisional Patent Application Ser. No. 60/180,002, filed Feb. 3, 2000 and incorporated herein by reference in its entirety.

### TECHNICAL FIELD

The present invention relates to medical devices for implantation in a body vessel. More particularly, the present invention relates to implantable medical device frames comprising a metallic bioabsorbable material, such as magnesium.

### BACKGROUND

Various implantable medical devices are advantageously inserted within various body vessels, for example from an implantation catheter. Minimally invasive techniques and instruments for placement of intraluminal medical devices have been developed to treat and repair such undesirable conditions within body vessels, including treatment of venous valve insufficiency. Intraluminal medical devices can be introduced to a point of treatment within a body vessel using a delivery catheter device passed through the vasculature communicating between a remote introductory location and the implantation site, and released from the delivery catheter device at the point of treatment within the body vessel. Intraluminal medical devices can be deployed in a vessel at a point of treatment, the delivery device withdrawn from the vessel, and the medical device retained within the vessel to provide sustained improvement in vascular valve function or to increase vessel patency.

Implantable medical devices typically comprise a support frame. The support frame, or portions thereof, can advantageously comprise a bioabsorbable material for some applications. Including a bioabsorbable material in the support frame can allow for the decomposition or absorption of all or part of the support frame during a period subsequent to implantation in a body vessel. A bioabsorbable support frame can be used, for example, to avoid future surgical extraction of an implant that serves a temporary function or to provide a medical device with post-implantation properties, such as frame stiffness, that change with time as portions of the frame are absorbed.

Medical devices can further comprise material for modifying the flow of fluid through a body vessel, such as a valve surface or an occlusion surface, that is attached to a support frame. For example, an implantable medical device can function as a replacement venous valve, or restore native venous valve function by bringing incompetent valve leaflets into closer proximity. Such devices can comprise an expandable support frame configured for implantation in the lumen of a body vessel, such as a vein. Venous valve devices can further comprise features that provide a valve function, such as opposable leaflets. Implantable valve devices can comprise a support frame made from one or more bioabsorbable materials, and optionally include other bioabsorbable or non-bioabsorbable materials.

Medical devices for intraluminal implantation, including implantable valves and support frames, often comprise support frames designed to assume a compressed configuration for intraluminal delivery, and then open to an expanded configuration upon deployment at a point of treatment within a body vessel. Materials for the support frame can be selected to provide desired mechanical properties allowing for expansion of a medical device without compromising mechanical integrity after deployment in the expanded state. Typically, metal materials are used to provide support frames that are ductile and mechanically durable, but not bioabsorbable. On the other hand, a variety of polymer-based bioabsorbable materials often provide frames with reduced mechanical durability that are bioabsorbable. Recently, metal materials have been developed that are bioabsorbable while still providing some of the advantages of mechanical durability of metal support frames. For example, U.S. Pat. No. 6,287,332 to Bolz et al. discloses various combinations of metal materials that are absorbed upon implantation in a body vessel.

What is needed are medical devices having an expandable support frame and comprising a metallic bioabsorbable material. Preferably, the medical device is suitable for use in an implantable valve, such as a venous valve.

### SUMMARY

The invention relates to medical devices for implantation in a body vessel. More specifically, preferred embodiments of the invention relate to medical devices that include a frame comprising metallic bioabsorbable material.

Preferably, the metallic bioabsorbable material is selected from a first group consisting of: magnesium, titanium, zirconium, niobium, tantalum, zinc and silicon. Also provided are mixtures and alloys of metallic bioabsorbable materials, including those selected from the first group.

In some embodiments, the metallic bioabsorbable material can be an alloy of materials from the first group and a material selected from a second group consisting of: lithium, sodium, potassium, calcium, iron and manganese. Without being limited to theory, it is believed that the metallic bioabsorbable material from the first group may form a protective oxide coat upon exposure to blood or interstitial fluid. The material from the second group is preferably soluble in blood or interstitial fluid to promote the dissolution of an oxide coat. The bioabsorption rate, physical properties and surface structure of the metallic bioabsorbable material can be adjusted by altering the composition of the alloy. In addition, other metal or non-metal components, such as gold, may be added to alloys or mixtures of metallic bioabsorbable materials. Some preferred metallic bioabsorbable material alloy compositions include lithium-magnesium, sodium-magnesium, and zinc-titanium, which can optionally further comprise gold.

The frame itself, or any portion of the frame, can be made from one or more metallic bioabsorbable materials, and can further comprise one or more non-metallic bioabsorbable materials, as well as various non-bioabsorbable materials. The bioabsorbable material can be distributed throughout the entire frame, or any localized portion thereof, in various ways. In some embodiments, the frame can comprise a bioabsorbable material or a non-bioabsorbable material as a “core” material, which can be at least partially enclosed by other materials. The frame can also have multiple bioabsorbable materials stacked on all or part of the surface of a non-bioabsorbable core material. The frame can also comprise a surface area presenting both a bioabsorbable material and a non-bioabsorbable material.

In other embodiments, a medical device can comprise a frame and a material attached to the frame. In preferred embodiments, the material can form one or more valve leaflets. In some embodiments, the valve material or the support frame can comprise a remodelable material. For treatment of many conditions, it is desirable that implantable medical devices comprise remodelable material. Implanted remodelable material provides a matrix or support for the growth of new tissue thereon, and remodelable material is absorbed into the body in which the device is implanted. Common events during this remodeling process include: widespread neovascularization, proliferation of granulation mesenchymal cells, biodegradation/resorption of implanted remodelable material, and absence of immune rejection. By this process, autologous cells from the body can replace the remodelable portions of the medical device.

The frame may, in some embodiments, comprise a plurality of struts, which can be of any suitable structure or orientation. In some embodiments, the frame comprises a plurality of struts connected by alternating bends. For example, the frame can be a ring or annular tube member comprising a series of struts in a “zig-zag” pattern. The frame can also comprise multiple ring members with struts in a “zig-zag” pattern, for example by connecting the ring members end to end, or in an overlapping fashion. In some embodiments, the struts are substantially aligned along the surface of a tubular plane, and substantially parallel to the longitudinal axis of the support frame.

In a first frame embodiment, the medical device can comprise a frame formed by joining two or more “zig-zag” rings together end to end and may optionally further comprise one or more leaflets attached thereto.

In a second frame embodiment, the medical device can comprise a frame member shaped in a serpentine configuration having a plurality of bends defining two or more legs, and optionally including one or more leaflets attached to each leg. Preferably, the frame member can comprise a bioabsorbable material and the leaflet can be formed by a remodelable material attached to the frame.

In a third frame embodiment, the medical device can comprise a valve structure and an expandable support frame configured to provide a sinus region or pocket between a valve leaflet and the widest radial dimension of the support frame. Upon implantation in a body vessel, the sinus region can promote increased fluid flow to reduce stagnation of fluid from around the valve structure, or promote closure of leaflets in response to retrograde fluid flow. For example, the sinus region can be created by a radially enlarged intermediate region in a tubular frame, or by a flared end of the support frame.

In a fourth frame embodiment, the medical device can comprise a frame configured to guide attached leaflets into increased radial proximity from a distal to a proximal end of a frame.

In some embodiments, the frame provides a first compliance in a first direction, and a material responsive to conditions within a body vessel to increase the compliance of the frame along the first direction. Absorption of a biomaterial can also increase the compliance of the frame in a first direction, for example by reducing the cross section or surface area of a portion of the frame. The absorption of the bioabsorbable material can also allow for the controlled fracture of a portion of the frame, resulting in a sudden change in the compliance of the frame.

In other embodiments, the medical device frame can include a cross section that can substantially conform to body vessel shapes that have elliptical or circular cross sections, and can change shape in response to changes in the cross section of a body vessel. The expanded configuration can have any suitable cross-sectional configuration, including circular or elliptical.

The medical device frame can also, in some embodiments, be characterized by a first radial compressibility along a first radial direction that is less than a second radial compressibility along a second direction.

Also provided are embodiments wherein the frame comprises a means for orienting the frame within a body vessel lumen. For example, the frame can comprise a marker, or a delivery device comprising the frame can provide indicia relating to the orientation of the frame within the body vessel.

In some embodiments, the medical device can comprise a frame and a means for regulating fluid through a body vessel. In some embodiments, the fluid can flow through the frame, while other embodiments provide for fluid flow through a lumen defined by the frame. Some embodiments comprise a frame and a first valve member connected to the frame. The valve member can be made from any suitable material, including a remodelable material or a synthetic polymer material. A valve member, according to some embodiments, can comprise a leaflet having a free edge responsive to the flow of fluid through the body vessel. For example, one or more valve members attached to a frame may, in one embodiment, permit fluid to flow through a body vessel in a first direction while substantially preventing fluid flow in the opposite direction. In some embodiments, the valve member comprises an extracellular matrix material, such as small intestine submucosa (SIS).

The medical devices of some embodiments can be expanded from a compressed delivery configuration to an expanded deployment configuration. Medical devices can be delivered intraluminally, for example using various types of delivery catheters, and expanded by conventional methods such as balloon expansion or self-expansion.

Also provided are embodiments wherein the frame comprises a means for orienting the frame within a body lumen. For example, the frame can comprise a marker, or a delivery device comprising the frame can provide indicia relating to the orientation of the frame within the body vessel.

Other embodiments provide methods of making medical devices described herein. Still other embodiments provide methods of treating a subject, which can be animal or human, comprising the step of implanting one or more support frames as described herein.

Other methods further comprise the step of implanting one or more frames attached to one or more valve members. In some embodiments, methods of treating may also include the step of delivering a medical device to a point of treatment in a body vessel, or deploying a medical device at the point of treatment.

Methods for treating certain conditions are also provided, such as venous valve insufficiency, varicose veins, esophageal reflux, restenosis or atherosclerosis.

Methods for delivering a medical device as described herein to any suitable body vessel are also provided, such as a vein, artery, biliary duct, ureteral vessel, body passage or portion of the alimentary canal. In some embodiments, medical devices having a frame with a compressed delivery configuration with a very low profile, small collapsed diameter and great flexibility, may be able to navigate small or tortuous paths through a variety of body vessels. A low-profile medical device may also be useful in coronary arteries, carotid arteries, vascular aneurysms, and peripheral arteries and veins (e.g., renal, iliac, femoral, popliteal, sublavian, aorta, intercranial, etc.). Other nonvascular applications include gastrointestinal, duodenum, biliary ducts, esophagus, urethra, reproductive tracts, trachea, and respiratory (e.g., bronchial) ducts. These applications may optionally include a sheath covering the medical device.

The invention includes other embodiments within the scope of the claims, and variations of all embodiments, and is limited only by the claims made by the Applicants. Additional understanding of the invention can be obtained by referencing the detailed description of embodiments of the invention, below, and the appended drawings.

### BRIEF DESCRIPTION OF THE DRAWINGS

FIG. 1 is a diagram of a “zig-zag” frame embodiment of the invention.

FIG. 2A is a diagram of a first medical device frame shown in the unfolded configuration; FIG. 2B shows the same medical device frame in the folded serpentine configuration within a body vessel. FIG. 2C shows another medical device frame having a serpentine configuration comprising a pair of legs. FIG. 2D shows fluid flowing through a medical device frame further comprising two leaflets; FIG. 2E shows the closure of two leaflets of a medical device in response to retrograde flow in a body vessel. FIG. 2F is a diagram of another medical device frame shown in a planar, unfolded configuration. FIG. 2G shows the medical device of FIG. 2F in a folded configuration within a body vessel.

FIG. 3A, FIG. 3B, FIG. 3C, and FIG. 3D are schematic views of illustrative embodiments of medical devices comprising a valve structure and a frame that creates an artificial sinus region adjacent to the valve leaflets.

FIG. 4, FIG. 5 and FIG. 6 are cross-section diagrams of exemplary frame embodiments comprising attachment regions that promote increased leaflet radial proximity between the distal and proximal ends of the frame.

### DETAILED DESCRIPTION OF EMBODIMENTS OF THE INVENTION

The following detailed description and appended drawings describe and illustrate various exemplary embodiments of the invention. The description and drawings serve to enable one skilled in the art to make and use the invention.

The invention provides medical devices for implantation in a body vessel which comprise a metallic bioabsorbable material, methods of making the medical devices, and methods of treatment that utilize the medical devices.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

The invention relates to medical devices for implantation in a body vessel. More specifically, preferred embodiments of the invention relate to medical devices that include a frame comprising metallic bioabsorbable material.

A large number of different types of materials are known in the art which may be inserted within the body and later dissipate. The term “bioabsorbable” is used herein to refer to materials selected to dissipate upon implantation within a body, independent of which mechanisms by which dissipation can occur, such as dissolution, degradation, absorption and excretion. The actual choice of which type of materials to use may readily be made by one ordinarily skilled in the art. Such materials are often referred to by different terms in the art depending upon the mechanism by which the material dissipates, as “bioabsorbable,” “bioabsorbable,” or “biodegradable.” The prefix “bio” indicates that the dissipation occurs under physiological conditions, as opposed to other processes, caused, for example, by UV light or weather conditions. The terms “bioresorption” and “bioabsorption” can be used interchangeably and refer to the ability of the polymer or its degradation products to be removed by biological events, such as by fluid transport away from the site of implantation or by cellular activity (e.g., phagocytosis). There may be some discussion among those skilled in the art as to the precise meaning and function of bioabsorbable materials, and how they differ from absorbable, absorbable, bioabsorbable, and biodegradable materials. Notwithstanding, the current disclosure contemplates all of these materials as “bioabsorbable” materials, as the aforementioned terminology is widely used interchangeably by medical professionals. Accordingly, and for conciseness of presentation, only the term “bioabsorbable” will be used in the following description to encompass absorbable, absorbable, bioabsorbable, and biodegradable, without implying the exclusion of the other classes of materials.

“Non-bioabsorbable” material refers to a material, such as a polymer or copolymer, which remains in the body without substantial bioabsorption.

As used herein, the term “body vessel” means any body passage lumen that conducts fluid, including but not limited to blood vessels, esophageal, intestinal, billiary, urethral and ureteral passages.

The term “alloy” refers to a substance composed of two or more metals or of a metal and a nonmetal intimately united, for example by chemical or physical interaction. Alloys can be formed by various methods, including being fused together and dissolving in each other when molten, although molten processing is not a requirement for a material to be within the scope of the term “alloy.” As understood in the art, an alloy will typically have physical or chemical properties that are different from its components.

The term “mixture” refers to a combination of two or more substances in which each substance retains its own chemical identity and properties.

The terms “frame” and “support frame” are used interchangeably herein to refer to a structure that can be implanted, or adapted for implantation, within the lumen of a body vessel.

Metallic Bioabsorbable Materials

Preferably, the metallic bioabsorbable material is selected from a first group consisting of: magnesium, titanium, zirconium, niobium, tantalum, zinc and silicon. Also provided are mixtures and alloys of metallic bioabsorbable materials, including those selected from the first group. Various alloys of the materials in the first group can also be used as a metallic bioabsorbable material, such as a zinc-titanium alloy, for example, as discussed in U.S. Pat. No. 6,287,332 to Bolz et al.

The physical properties of the alloy can be controlled by selecting the metallic bioabsorbable material, or forming alloys of two or more metallic bioabsorbable materials. For example, the percentage by weight of titanium can be in the range of 0.1% to 1%, which can reduce the brittle quality of crystalline zinc. Without being bound to theory, it is believed that the addition of titanium leads to the formation of a Zn₁₅ Ti phase. In another embodiment, gold can be added to the zinc-titanium alloy at a percentage by weight of 0.1% to 2%, resulting in a further reduction of the grain size when the material cures and further improving the tensile strength of the material. These materials can be incorporated in the support frame of a medical device, including a venous valve frame.

In some embodiments, the metallic bioabsorbable material can be an alloy of materials from the first group and a material selected from a second group consisting of: lithium, sodium, potassium, calcium, iron and manganese. The metallic bioabsorbable material from the first group can form a protective oxide coating upon exposure to blood or interstitial fluid. The material from the second group is preferably soluble in blood or interstitial fluid to promote the dissolution of the oxide coating. Also provided are mixtures and alloys of metallic bioabsorbable materials, including those selected from the second group and combinations of materials from the first group and the second group.

Further details relating to these metallic bioabsorbable materials are found in U.S. Pat. No. 6,287,332 to Bolz et al., which is incorporated herein by reference in its entirety.

Preferably, the support frame comprises magnesium or an alloy thereof. U.S. Pat. No. 6,287,332 to Bolz et al. provides examples of materials suitable for medical device support frames, which are incorporated herein by reference. For example, in one embodiment, the metallic bioabsorbable material comprises an alloy of lithium and magnesium with a magnesium-lithium ratio of about 60:40. The fatigue durability of the lithium:magnesium alloy can optionally be increased by the addition of further components such as zinc. In another embodiment, the medical device support frame comprises a sodium-magnesium alloy.

The frame itself, or any portion of the frame, can be made from one or more metallic bioabsorbable materials, and can further comprise one or more non-metallic bioabsorbable materials, as well as various non-bioabsorbable materials. The bioabsorbable material can be distributed throughout the entire frame, or any localized portion thereof, in various ways. In some embodiments, the frame can comprise a bioabsorbable material or a non-bioabsorbable material as a “core” material, which can be at least partially enclosed by other materials. The frame can also have multiple bioabsorbable materials stacked on all or part of the surface of a non-bioabsorbable core material. The frame can also comprise a surface area presenting both a bioabsorbable material and a non-bioabsorbable material.

Other Bioabsorbable Materials

In addition to a metallic bioabsorbable material, the frame can further comprise a bioabsorbable material, selected from any number of bioabsorbable homopolymers, copolymers, or blends of bioabsorbable polymers. In some embodiments, a medical device frame can comprise a biocompatible, bioabsorbable polymer or copolymer; a synthetic, biocompatible, non-bioabsorbable polymer or copolymer; or combinations thereof.

During the last 20 to 30 years, several bioabsorbable, biocompatible polymers have been developed for use in medical devices, and have been approved for use by the U.S. Food and Drug Administration (FDA). These FDA-approved materials include polyglycolic acid (PGA), polylactic acid (PLA), Polyglactin 910 (comprising a 9:1 ratio of glycolide per lactide unit, and known also as VICRYL™), polyglyconate (comprising a 9:1 ratio of glycolide per trimethylene carbonate unit, and known also as MAXON™), and polydioxanone (PDS). In general, these materials biodegrade in vivo in a matter of months, although some more crystalline forms can biodegrade more slowly. These materials have been used in orthopedic applications, wound healing applications, and extensively in sutures after processing into fibers. More recently, some of these polymers also have been used in tissue engineering applications.

A variety of bioabsorbable and biocompatible materials can be used to make medical device frames useful with particular embodiments disclosed herein, depending on the combination of properties desired. Properties such as flexibility, compliance, and rate of bioabsorption can be selected by choosing appropriate bioabsorbable materials. The properties of the bioabsorbable polymers may differ considerably depending on the nature and amounts of the comonomers, if any, employed and/or the polymerization procedures used in preparing the polymers.

Biodegradable polymers that can be used to form the support frame of a medical device, or can be coated on a frame, include a wide variety of materials. Examples of such materials include polyesters, polycarbonates, polyanhydrides, poly(amino acids), polyimines, polyphosphazenes and various naturally occurring biomolecular polymers, as well as co-polymers and derivatives thereof. Certain hydrogels, which are cross-linked polymers, can also be made to be biodegradable. These include, but are not necessarily limited to, polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, poly-alpha-hydroxy acids, trimethlyene carbonate, poly-beta-hydroxy acids, polyorganophosphazines, polyanhydrides, polyesteramides, polyethylene oxide, polyester-ethers, polyphosphoester, polyphosphoester urethane, cyanoacrylates, poly(trimethylene carbonate), poly(iminocarbonate), polyalkylene oxalates, polyvinylpyrolidone, polyvinyl alcohol, poly-N-(2-hydroxypropyl)-methacrylamide, polyglycols, aliphatic polyesters, poly(orthoesters), poly(ester-amides), polyanhydrides, modified polysaccharides and modified proteins. Some specific examples of bioabsorbable materials include poly(epsilon-caprolactone), poly(dimethyl glycolic acid), poly(hydroxy butyrate), poly(p-dioxanone), polydioxanone, PEO/PLA, poly(lactide-co-glycolide), poly(hydroxybutyrate-co-valerate), poly(glycolic acid-co-trimethylene carbonate), poly(epsilon-caprolactone-co-p-dioxanone), poly-L-glutamic acid or poly-L-lysine, polylactic acid, polylactide, polyglycolic acid, polyglycolide, poly(D,L-lactic acid), L-polylactic acid, poly(glycolic acid), polyhydroxyvalerate, cellulose, chitin, dextran, fibrin, casein, fibrinogen, starch, collagen, hyaluronic acid, hydroxyethyl starch, and gelatin.

In some embodiments, the frame or coatings thereon comprise a degradable polyesters, such as a poly(hydroxyalkanoates), for example poly(lactic acid) (polylactide, PLA), poly(glycolic acid) (polyglycolide, PGA), poly(3-hydroxybutyrate), poly(4-hydroxybutyrate), poly(3-hydroxyvalerate), and poly(caprolactone), or poly(valerolactone). Useful biodegradable polycarbonates include poly(trimethylene carbonate), poly(1,3-dioxan-2-one), poly(p-dioxanone), poly(6,6-dimethyl-1,4-dioxan-2-one), poly(1,4-dioxepan-2-one), and poly(1,5-dioxepan-2-one).

Other examples of degradable polymers that can be used in or on the frame include polyorthoesters, polyorthocarbonates, polyoxaesters (including poly(ethylene oxalate) and poly(alkylene oxalates)), polyanhydrides, poly(amino acids) such as polylysine, polyimines such as poly(ethylene imine) (PEI), poly(iminocarbonates), and biodegradable polyphosphazenes such as poly(phenoxy-co-carboxylatophenoxy phosphazene).

Certain naturally occurring polymers can also be used in or on the frame, including: fibrin, fibrinogen, elastin, collagens, chitosan, extracellular matrix (ECM), carrageenan, chondroitin, pectin, alginate, alginic acid, albumin, dextrin, dextrans, gelatins, mannitol, n-halamine, polysaccharides, poly-1,4-glucans, starch, hydroxyethyl starch (HES), dialdehyde starch, glycogen, amylase, hydroxyethyl amylase, amylopectin, glucoso-glycans, fatty acids (and esters thereof), hyaluronic acid, protamine, polyaspartic acid, polyglutamic acid, D-mannuronic acid, L-guluronic acid, zein and other prolamines, alginic acid, guar gum, and phosphorylcholine, as well as co-polymers and derivatives thereof.

Various cross linked polymer hydrogels can also be used in forming the frame or coating the frame. The hydrogel can be formed, for example, using a base polymer selected from any suitable polymer, preferably poly(hydroxyalkyl (meth)acrylates), polyesters, poly(meth)acrylamides, poly(vinyl pyrollidone) and poly(vinyl alcohol). A cross-linking agent can be one or more of peroxides, sulfur, sulfur dichloride, metal oxides, selenium, tellurium, diamines, diisocyanates, alkyl phenyl disulfides, tetraalkyl thiuram disulfides, 4,4′-dithiomorpholine, p-quinine dioxime and tetrachloro-p-benzoquinone. Also, boronic acid-containing polymer can be incorporated in hydrogels, with optional photopolymerizable group, into degradable polymer, such as those listed above.

Finally, various bioactive coating compounds can be incorporated on or in the support frame. Examples of bioactive coating compounds include antibodies, such as EPC cell marker targets, CD34, CD133, and AC 133/CD133; Liposomal Biphosphate Compounds (BPs), Chlodronate, Alendronate, Oxygen Free Radical scavengers such as Tempamine and PEA/NO preserver compounds, and an inhibitor of matrix metalloproteinases, MMPI, such as Batimastat. Still other bioactive agents that can be incorporated in or coated on a frame include a PPARα-agonist, a PPAR δ agonist and RXR agonists, as disclosed in published U.S. Patent Application U.S. 2004/0073297 to Rohde et al., published on Apr. 15, 2004 and incorporated in its entirety herein by reference.

The frame can comprise or be coated with polysaccharides, for example as disclosed in published U.S. Patent Application U.S. 2004/091605 to Bayer et al., published on May 13, 2004 and incorporated herein by reference in its entirety. In one embodiment, the frame comprises a polysaccharide layer which has improved adhesion capacity on the substrate surface of the frame. For example, the coated frame can comprise the covalent bonding of a non-crosslinked hyaluronic acid to a substrate surface of the frame with the formation of hyaluronic acid layer and crosslinking of the hyaluronic acid layer.

Copolymers of degradable polymers may also be used, as well as copolymers of degradable and biostable polymers. These copolymers may be formed by copolymerization of compatible monomers or by linking or copolymerization of functionalized chains with other functionalized chains or with monomers. Examples include crosslinked phosphorylcholine-vinylalkylether copolymer and PC-Batimastat copolymers.

In one embodiment, the frame is coated with a polymeric coating of between about 1 μm and 50 μm, or preferably between 3 μm and 30 μm, although any suitable thickness can be selected. The coating can be biologically or chemically passive or active.

Other Frame Components

In addition to a metallic bioabsorbable metal, the support frame can comprise other metal or non-metal materials. In some embodiments, portions of a support frame can comprise a core layer of a base material surrounded or partially covered by a bioabsorbable metallic material.

Examples of materials that can be used to form a frame, or can be coated on a frame, include biocompatible metals or other metallic materials; polymers including bioabsorbable or biostable polymers; stainless steels (e.g., 316, 316L or 304); nickel-titanium alloys including shape memory or superelastic types (e.g., nitinol or elastinite); noble metals including platinum, gold or palladium; refractory metals including tantalum, tungsten, molybdenum or rhenium; stainless steels alloyed with noble and/or refractory metals; silver; rhodium; inconel; iridium; niobium; titanium; magnesium; amorphous metals; plastically deformable metals (e.g., tantalum); nickel-based alloys (e.g., including platinum, gold and/or tantalum alloys); iron-based alloys (e.g., including platinum, gold and/or tantalum alloys); cobalt-based alloys (e.g., including platinum, gold and/or tantalum alloys); cobalt-chrome alloys (e.g., elgiloy); cobalt-chromium-nickel alloys (e.g., phynox); alloys of cobalt, nickel, chromium and molybdenum (e.g., MP35N or MP20N); cobalt-chromium-vanadium alloys; cobalt-chromium-tungsten alloys; platinum-iridium alloys; platinum-tungsten alloys; magnesium alloys; titanium alloys (e.g., TiC, TiN); tantalum alloys (e.g., TaC, TaN); L605; magnetic ferrite; nonmetallic biocompatible materials including polyamides, polyolefins (e.g., polypropylene or polyethylene), nonabsorbable polyesters (e.g., polyethylene terephthalate) or bioabsorbable aliphatic polyesters (e.g., homopolymers or copolymers of lactic acid, glycolic acid, lactide, glycolide, para-dioxanone, trimethylene carbonate or .epsilon.-caprolactone); polymeric materials (e.g., poly-L-lactic acid, polycarbonate, polyethylene terephthalate or engineering plastics such as thermotropic liquid crystal polymers (LCPs)); biocompatible polymeric materials (e.g., cellulose acetate, cellulose nitrate, silicone, polyethylene terephthalate, polyurethane, polyamide, polyester, polyorthoester, polyanhydride, polyether sulfone, polycarbonate, polypropylene, high molecular weight polyethylene or polytetrafluoroethylene); degradable or biodegradable polymers, plastics, natural (e.g., animal, plant or microbial) or recombinant material (e.g., polylactic acid, polyglycolic acid, polyanhydride, polycaprolactone, polyhydroxybutyrate valerate, polydepsipeptides, nylon copolymides, conventional poly(amino acid) synthetic polymers, pseudo-poly(amino acids) or aliphatic polyesters (e.g., polyglycolic acid (PGA), polylactic acid (PLA), polyalkylene succinates, polyhydroxybutyrate (PHB), polybutylene diglycolate, poly epsilon-caprolactone (PCL), polydihydropyrans, polyphosphazenes, polyorthoesters, polycyanoacrylates, polyanhydrides, polyketals, polyacetals, poly(.alpha.-hydroxy-esters), poly(carbonates), poly(imino-carbonates), poly(.beta.-hydroxy-esters) or polypeptides)); polyethylene terephthalate (e.g., dacron or mylar); expanded fluoropolymers (e.g., polytetrafluoroethylene (PTFE)); fluorinated ethylene propylene (FEP); copolymers of tetrafluoroethylene (TFE) and per fluoro(propyl vinyl ether) (PFA)); homopolymers of polychlorotrifluoroethylene (PCTFE) and copolymers with TFE; ethylene-chlorotrifluoroethylene (ECTFE); copolymers of ethylene-tetrafluoroethylene (ETFE); polyvinylidene fluoride (PVDF); polyvinyfluoride (PVF); polyaramids (e.g., kevlar); polyfluorocarbons including polytetrafluoroethylene with and without copolymerized hexafluoropropylene (e.g., teflon or goretex); expanded fluorocarbon polymers; polyglycolides; polylactides; polyglycerol sebacate; polyethylene oxide; polybutylene terepthalate; polydioxanones; proteoglycans; glycosaminoglycans; poly(alkylene oxalates); polyalkanotes; polyamides; polyaspartimic acid; polyglutarunic acid polymer; poly-p-diaxanone (e.g., PDS); polyphosphazene; polyurethane including porous or nonporous polyurethanes; poly(glycolide-trimethylene carbonate); terpolymer (copolymers of glycolide, lactide or dimethyltrimethylene carbonate); polyhydroxyalkanoates (PHA); polyhydroxybutyrate (PHB) or poly(hydroxybutyrate-co-valerate) (PHB-co-HV); poly(epsilon-caprolactone) (e.g., lactide or glycolide); poly(epsilon-caprolactone-dimethyltrimethylene carbonate); polyglycolic acid (PGA); poly-L and poly-D(lactic acid) (e.g., calcium phosphate glass); lactic acid/ethylene glycol copolymers; polyarylates (L-tyrosine-derived) or free acid polyarylates; polycarbonates (tyrosine or L-tyrosine-derived); poly(ester-amides); poly(propylene fumarate-co-ethylene glycol) copolymer (e.g., fumarate anhydrides); polyanhydride esters; polyanhydrides; polyorthoesters; prolastin or silk-elastin polymers (SELP); calcium phosphate (bioglass); compositions of PLA, PCL, PGA ester; polyphosphazenes; polyamino acids; polysaccharides; polyhydroxyalkanoate polymers; various plastic materials; teflon; nylon; block polymers or copolymers; Leica RM2165; Leica RM2155; organic fabrics; biologic agents (e.g., protein, extracellular matrix component, collagen, fibrin); small intestinal submucosa (SIS) (e.g., vacuum formed SIS); collagen or collagen matrices with growth modulators; aliginate; cellulose and ester; dextran; elastin; fibrin; gelatin; hyaluronic acid; hydroxyapatite; polypeptides; proteins; ceramics (e.g., silicon nitride, silicon carbide, zirconia or alumina); bioactive silica-based materials; carbon or carbon fiber; cotton; silk; spider silk; chitin; chitosan (NOCC or NOOC-G); urethanes; glass; silica; sapphire; composites; any mixture, blend, alloy, copolymer or combination of any of these; or various other materials not limited by these examples.

In some embodiments, a frame comprises a core or “base” material surrounded by, or combined, layered, or alloyed with a metallic bioabsorbable material.

In one embodiment, the frame can comprise silicon-carbide (SiC). For example, published U.S. Patent Application No. U.S. 2004/034409 to Hueblein et al., published on Feb. 14, 2004 and incorporated in its entirety herein by reference, discloses various suitable frame materials and configurations.

Support Frame and Valve Embodiments

The frame may, in some embodiments, comprise a plurality of struts, which can be of any suitable structure or orientation. In one embodiment, the frame comprises a plurality of struts connected by alternating bends. For example, the frame can be a sinusoidal ring member comprising a series of struts in a “zig-zag” pattern. The frame can also comprise multiple ring members with struts in a “zig-zag” pattern, for example by connecting the ring members end to end, or in an overlapping fashion. In some embodiments, the struts are substantially aligned along the surface of a tubular plane, substantially parallel to the longitudinal axis of the support frame.

Certain non-limiting examples of frame embodiments are provided herein to illustrate selected features of the medical devices relating to component frames. Medical devices can comprise the frame embodiments discussed below, and combinations, variations or portions thereof, as well as other frame configurations. Medical devices comprising various frames in combination with material suitable to form a leaflet attached thereto are also within the scope of some embodiments of the invention.

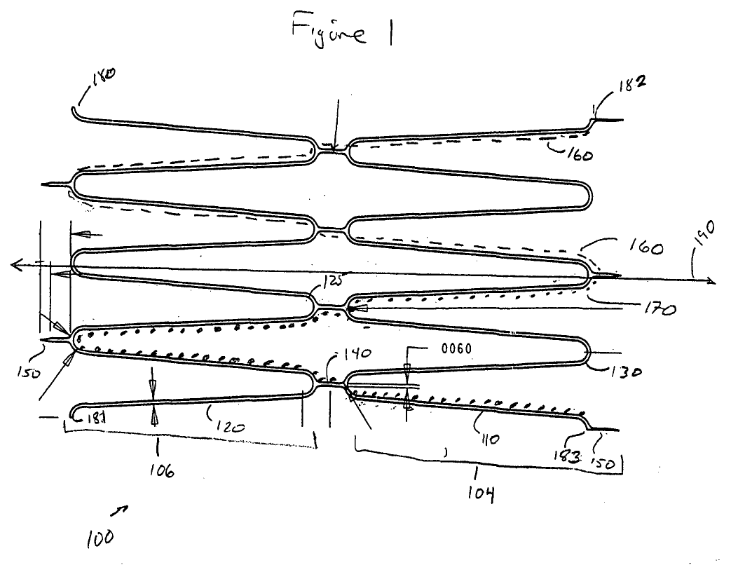

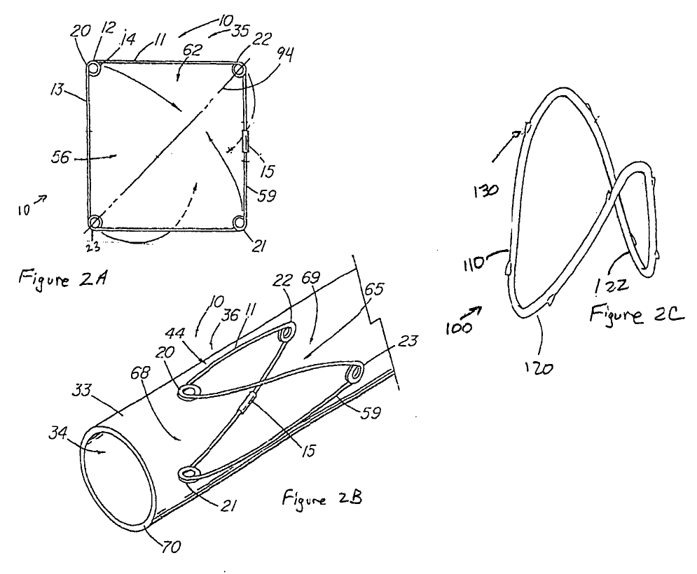

In a first frame embodiment, the medical device can comprise a frame formed by joining two or more “zig-zag” rings together end to end and optionally attaching valve leaflet material thereto. FIG. 1 is a diagram of a “zig-zag” frame embodiment of the invention. The frame 100 is shown in a flat configuration. The frame 100 can be folded into a tubular comfiguration by joining a first proximal point 180 to a second proximal point 181, and a first distal point 182 to a second distal point 183. In the folded tubular configuration, the frame 100 comprises a first ring 106 formed from a plurality of interconnected struts 120 in an alternating configuration connected by a series of bends 125. The first ring 106 is joined to a second ring 104 by a series of interconnecting struts 140. The second ring 104 also comprises a plurality of interconnected struts 110 in an alternating configuration, connected by a series of bends 130. In this embodiment, certain bends comprise an integral barb 150 formed by a pointed extension of the frame material away from the interconnecting struts 140. The barb 150 can engage the interior wall of a body vessel to anchor the medical device upon intraluminal implantation. While the illustrated embodiment shows a frame 100 having a first ring 106 and a second ring 104, other embodiments may comprise one or more rings. The frame may comprise two or more rings joined together along a longitudinal axis (as shown in frame 100) or along a transverse axis. Multiple rings may be joined by any number of interconnecting struts, or directly fused, without interconnecting struts. The struts of the frame may have any suitable shape, and may include perforations, ridges, and rough or smooth surfaces.

In the folded tubular configuration, the frame 100 has a longitudinal axis 190 and defines a tubular interior lumen area surrounded by the frame 100. Preferably, the frame 100 is implanted in a tubular configuration within a body vessel such that the longitudinal axis 190 of the frame is substantially aligned with the longitudinal axis of the body vessel. The frame 100 in the tubular configuration can be compressed to a low-profile delivery configuration, delivered to a point of treatment within a body vessel, and expanded (for example, by self-expansion or balloon expansion) during deployment. The frame 100 can also optionally comprise one or more valve leaflets to regulate fluid flow through the lumen of the frame. A first leaflet can be attached to the frame 100 along a first attachment path 160. An optional second leaflet can be attached to the frame 100 along a second attachment path 170.

In a second frame embodiment, the medical device can comprise a frame member shaped in a serpentine configuration having a plurality of bends defining two or more legs, with a leaflet attached to each leg. Examples of such frames are provided in U.S. Pat. Nos. 6,508,833 and 6,200,336 to Pavcnik, and U.S. patent application Ser. Nos. 10/721,582, filed Nov. 25, 2003; Ser. No. 10/642,372, filed Aug. 15, 2003; and Ser. No. 10/294,987, filed Nov. 14, 2002, all of which are incorporated herein by reference in their entirety. Preferably, the frame member can comprise a bioabsorbable material and the leaflet can be formed by a remodelable material attached to the frame.

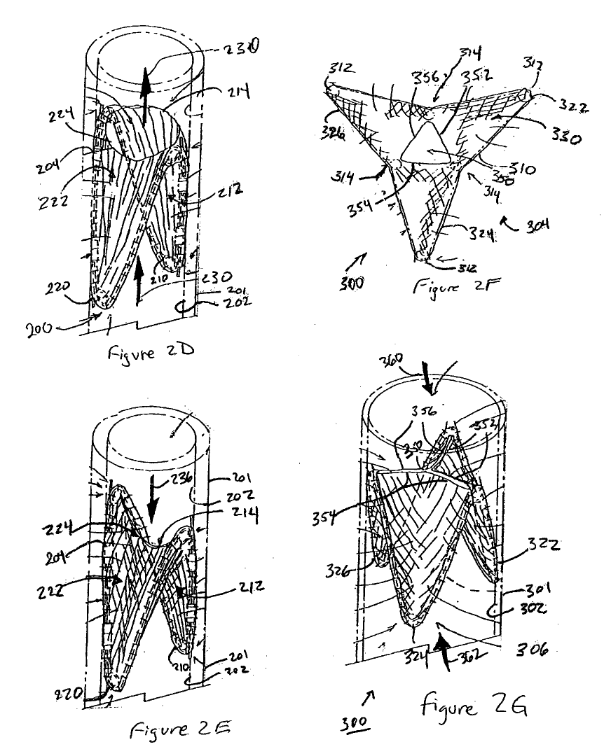

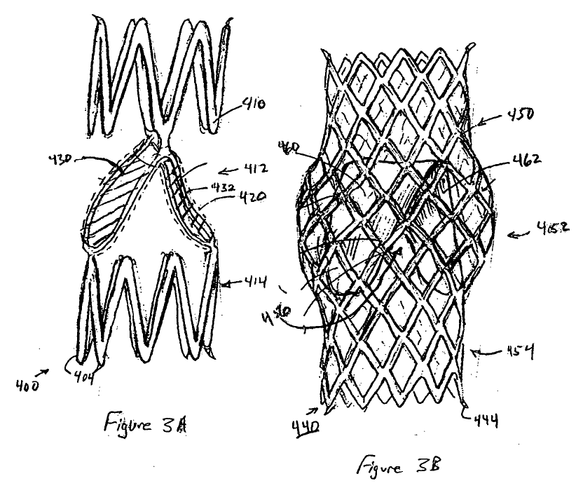

FIG. 2A is a first medical device frame shown in a planar, unfolded configuration. The medical device comprises a frame 10 formed from a closed circumference 62 of a single piece 59 of material that is formed into a device 10 having a plurality of sides 13 interconnected by a series of bends 12. The depicted embodiment includes four sides 13 of approximately equal length. Alternative embodiments include forming a frame into any polygonal shape, for example a pentagon, hexagon, octagon, etc. The bends 12 interconnecting the sides 13 can optionally comprise a coil 14 of approximately one and a quarter turns, or can be formed into a fillet comprising a series of curves, or simply consist of a single curve in a straight wire frame piece 59. The device 10 depicted in FIG. 2A is shown in its first configuration 35 whereby all four bends 20, 21, 22, 23 and each of the sides 13 generally lie within a single flat plane.

FIG. 2B shows the medical device frame of FIG. 2A in a folded serpentine configuration within a body vessel. To resiliently reshape the device 10 into a second configuration 36, shown in FIG. 2B, the frame 10 of FIG. 2A is folded twice, first along one diagonal axis with opposite bends 20 and 21 being brought into closer proximity, followed by opposite bends 22 and 23 being folded together and brought into closer proximity in the opposite direction. The second configuration 36, depicted in FIG. 2B, has two opposite bends 20, 21 oriented at the first end 68 of the device 10, while the other opposite bends 22, 23 are oriented at the second end 69 of the device 10 and rotated approximately 90 degrees with respect to bends 20 and 21 when viewed in cross section. The medical device in the second configuration 36 can be used as a stent 44 to maintain an open lumen 34 in a vessel 33, such as a vein, artery, or duct.

FIG. 2C shows a second medical device support frame. The support frame 100 comprises a continuous member 110 shaped into a serpentine configuration that defines a first leg 120 and a second leg 122. The member 110 can optionally comprise one or more barbs 130 extending as pointed protrusions from the member 110.

FIG. 2D shows fluid flowing through a medical device frame further comprising two leaflets. The medical device 200 is implanted within a lumen 202 of a body vessel 201. The medical device comprises a support frame 204 in a serpentine configuration having a first leg 210 and a second leg 220. Examples of suitable support frames are shown in FIGS. 2A-2C. A first leaflet 212 is attached to the first leg 210, and a second leaflet 222 is attached to the first leg 212, by any suitable means along the edges of portions of each leg of the frame. An unattached portion of the first leaflet 212 forms a first free edge 214; and an unattached portion of the second leaflet 222 forms a second free edge 224. The first free edge 214 and the second free edge 224 together define a valve orifice that allows fluid to flow in one direction, while substantially preventing fluid flow in an opposite, retrograde direction. When fluid flows in a first direction 230, the fluid forces the first free edge 214 and the second free edge 224 open to permit continued fluid flow through the valve. However, as shown in FIG. 2E, when the valve 200 is subjected to retrograde flow, the valve orifice closes as the first free edge 214 and the second free edge 224 cooperatively close across the lumen 202 of the body vessel 201.

Other medical device embodiments can have different numbers and arrangements of legs and leaflets. For example, the medical device can comprise one leaflet and two legs, or three or more legs and leaflets.

FIG. 2F shows a third medical device frame 300 shown in a planar, unfolded configuration 304. The medical device 300 comprises a support frame 310 with three sides joined by a first series of bends 312. A second series of bends 314 are positioned at the midpoints of each of the three sides. The three mid-point bends 314 are drawn radially toward the center, and the frame is held in this shape by a covering 330 attached to the frame. With the midpoint bends 314 held in the inwardly drawn configuration, for example by the attached covering 330, the frame 310 forms a first leg 322, a second leg 324 and a third leg 326. A portion of the covering 330 can be removed to define a valve orifice 350 inside the support frame 310. The edges of the valve orifice 350 are defined by a first free edge 352 along the first leg 322, a second free edge 354 along the second leg 324 and a third free edge 356 along a third leg 326.

FIG. 2G shows the medical device of FIG. 2F in a folded configuration 306 within the lumen 302 of a body vessel 301. The medical device 300 is as described in FIG. 2F above, except that the first leg 322, the second leg 324 and the third leg 326 are oriented along the longitudinal axis of the body vessel 301. The medical device 300 is subjected to fluid flow in a retrograde direction 360, the free edges close against one another to substantially inhibit retrograde flow through the valve orifice 350. More specifically, the first free edge 352, the second free edge 354 and the third free edge 356 cooperate to close the valve orifice 350 when subjected to fluid flow in the retrograde direction. However, the free edges are pressed open by fluid flow in the opposite direction 362, thereby opening the valve orifice 350.

In a third frame embodiment, the medical device can comprise a valve structure and an expandable support frame configured to provide an sinus region or pocket between a valve leaflet and the farthest radial dimension of the support frame. Examples of frames configured to provide a sinus region or pocket upon implantation in a body vessel are found in U.S. patent application Ser. No. 10/282,716, filed on Apr. 21, 2004 to Case et al., which is incorporated herein in its entirety. Upon implantation in a body vessel, the sinus region can promote increased fluid flow to reduce stagnation of fluid from around the valve structure, or to promote closure of leaflets in response to retrograde fluid flow. For example, the sinus region can be created by a radially enlarged intermediate region in a tubular frame, or by a flared proximal end of the support frame.

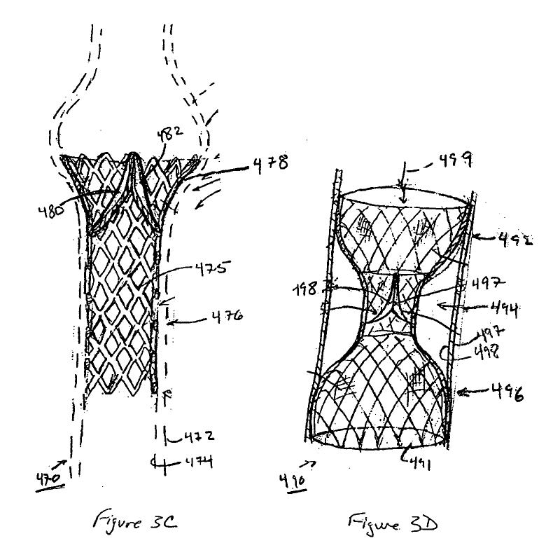

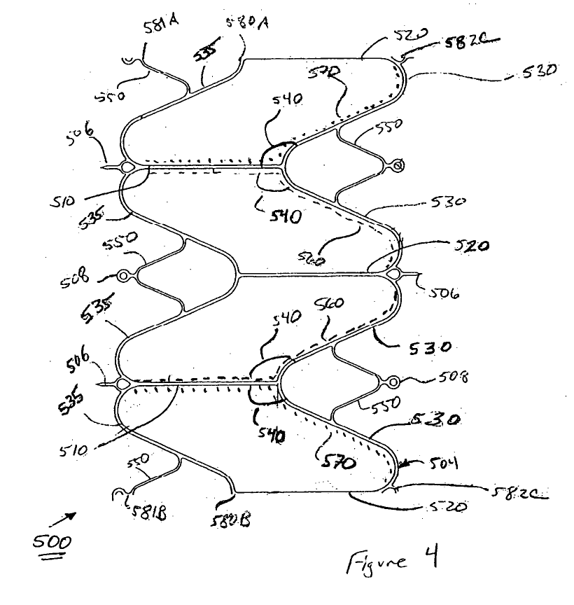

FIG. 3A, FIG. 3B, FIG. 3C, and FIG. 3D are schematic views of illustrative embodiments of medical devices comprising a valve structure and a frame that creates an artificial sinus region adjacent to the valve leaflets. A first medical device 400 is illustrated in FIG. 3A and comprises support frame 404 having a first end region 410 and a second end region 414 that are substantially identical, and are connected by an intermediate region 412. In the end regions, the support frame 404 comprises a plurality of alternating struts and bends arranged in a “zig-zag” pattern and joined into a ring. The support frame 404 in the intermediate region 412 comprises a sinusoidal configuration having two legs. A first leaflet 420 and a second leaflet 430 are joined to the support frame 404 in the intermediate region 412 along a line of attachment 432.

A second medical device 440 is illustrated in FIG. 3B. The medical device 440 comprises a frame 444 comprising a mesh of intersecting struts arranged in a tubular configuration. The frame 444 has a first end region 450 continuously joined to a radially expanded intermediate region 452 that is, in turn, continuously joined to a second end region 454 that has a cross sectional profile that mirrors that of the first end region 450. The flared portion of the intermediate region 452 creates an artificial sinus region 456 within the tubular structure. A first valve leaflet 460 and a second valve leaflet 462 are mounted to the support frame 444 within the sinus region 456.

A third medical device is illustrated in FIG. 3C. The medical device 470 comprises a tubular support frame 475 having a flared first end region 478 continuously joined to a second end region 476. The tubular frame 475 comprises a plurality of struts joined in a mesh and formed into a tube. The flared first end region 478 creates an artificial sinus region around a first valve leaflet 480 and a second valve leaflet 482 that are attached to the support frame 475.

A fourth medical device of the third frame embodiment is illustrated in FIG. 3D. The medical device 490 comprises a tubular support frame 491 made from a mesh-shaped plurality of interconnected struts, and has a radially narrowed intermediate region 494 continuously joined on each end to a first end region 492 and a second end region 496, respectively. A first valve leaflet 497 and a second valve leaflet 498 are mounted within the intermediate region 494 and oriented to prevent flow in a retograde direction 499 when the medical device 490 is implanted within the lumen 498 of a body vessel 497.

In a fourth frame embodiment, medical devices can comprise a frame configured to guide attached leaflets into increased radial proximity from the distal to the proximal end of the frame.

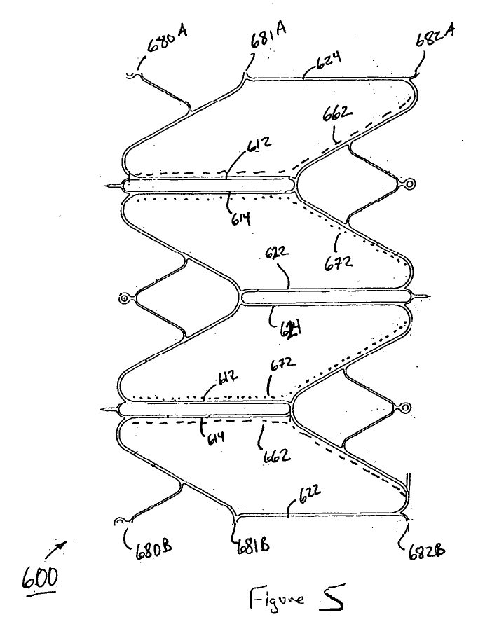

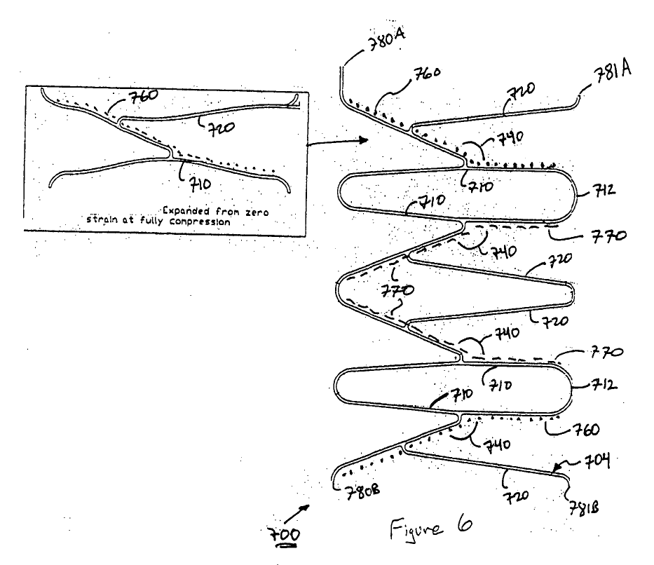

FIG. 4 is a cross section diagram of an exemplary frame embodiment comprising attachment regions promoting increased leaflet radial proximity between the distal and proximal ends of the frame. The medical device 500 comprises a support frame 504 that can be formed into a tubular configuration by attaching point 580A to point 580B, point 581A to point 581B and point 582A to point 582B. The support frame 504 comprises a series of alternating longitudinal attachment struts 510 and longitudinal support struts 520 joined at a distal end by a series of curved distal attachment struts 530 and joined at a proximal end by a series of curved proximal support struts 535. The frame 504 can also comprise one or more support arms 550 between adjacent distal attachment struts 530 or proximal support struts 535. The distal attachment struts 530 are joined to the longitudinal attachment struts 510 to form a first interior angle 540 that is preferably greater than 90-degrees and less than 180 degrees. The frame 504 can optionally comprise one or more barbs 506 or radiopaque markers 508. The medical device 500 can optionally comprise one or more leaflets. For example, a first leaflet can be attached to the frame 504 along a first attachment path 560, and a second leaflet can be attached to the frame 504 along a second attachment path 570.

FIG. 5 is a cross section diagram of another exemplary frame embodiment comprising attachment regions promoting increased leaflet radial proximity between the distal and proximal ends of the frame. As with the medical device 500 of FIG. 4, the medical device 600 comprises a support frame 604 that can be formed into a tubular configuration by attaching point 680A to point 680B, point 681A to point 681B and point 682A to point 682B.

The medical device 600 comprises a support frame 604 that is the same as the frame 504 illustrated in the medical device 500 of FIG. 4, except that the frame 604 comprises pairs of parallel longitudinal struts instead of single longitudinal attachment struts. More specifically, the support frame 604 comprises parallel sets of longitudinal attachment struts including a set of first longitudinal attachment struts 612 and a paired set of second longitudinal attachment struts 614. Similarly, the support frame also comprises parallel sets of longitudinal support struts including a set of first longitudinal support struts 622 and a paired set of second longitudinal support struts 624. The medical device 600 can optionally comprise one or more leaflets. For example, a first leaflet can be attached to the frame 604 along a first attachment path 662, and a second leaflet can be attached to the frame 604 along a second attachment path 672.

FIG. 6 is a cross section diagram of yet another exemplary frame comprising attachment regions promoting increased leaflet radial proximity between the distal and proximal ends of the frame. The medical device 700 comprises a support frame 704 that can be formed into a tubular configuration by attaching point 780A to point 780B, and point 781A to point 781B. The support frame 704 comprises a series of alternating longitudinal attachment struts 710 and longitudinal support struts 720 joined at a distal end by a series of curved distal attachment struts 730. The longitudinal attachment struts 710 are tapered between the point of attachment of the distal attachment struts 730 and adjacent pairs of longitudinal attachment struts 710 are attached at a common distal point 712. The distal attachment struts 730 are joined to the longitudinal attachment struts 710 to form a first interior angle 740 that is preferably greater than 90-degrees and less than 180 degrees. The medical device 700 can optionally comprise one or more leaflets. For example, a first leaflet can be attached to the frame 704 along a first attachment path 760, and a second leaflet can be attached to the frame 704 along a second attachment path 770.

Another frame suitable for use with medical devices comprises an array of interconnecting members defining T-shaped openings in a tubular frame, as disclosed in U.S. Pat. No. 6,613,080 to Lootz, issued on Sep. 3, 2003 and incorporated in its entirety herein by reference.

The medical devices of the embodiments described herein may be oriented in any suitable absolute orientation with respect to a body vessel. The recitation of a “first” direction is provided as an example. Any suitable orientation or direction may correspond to a “first” direction. The medical devices of the embodiments described herein may be oriented in any suitable absolute orientation with respect to a body vessel. For example, the first direction can be a radial direction in some embodiments.

In some embodiments, the invention provides frames with compliance that can vary with time, enabling one skilled in the art to design, make and use medical devices that provide desired levels of compliance at different time periods. Examples of such frames are provided in U.S. Provisional Patent Application 60/561,739, filed Apr. 13, 2004 by Case et al., which is incorporated herein by reference in its entirety. As discussed therein, “compliance” refers to the displacement of the body frame in response to a given force directed inward toward the center of the frame. Increased compliance is measured by comparing the frame displacement in response to the same force applied inward to the frame along the same direction at two different points in time. The increase in compliance of the frame upon implantation can occur in several ways. For example, a portion of a frame can be bioabsorbed or fracture in a controlled fraction to increase the frame compliance in a first direction. In some embodiments, the frame can comprise various materials or configurations to provide an increased compliance after a period of time after implantation.

Medical devices with variable compliance can provide, for example, an optimal amount of tension on an attached remodelable material during the remodeling process, and then provide increased compliance and minimal body vessel distortion after the remodeling process is completed provides a first compliance in a first direction, and a material responsive to conditions within a body vessel to increase the compliance of the frame along the first direction. Absorption of a biomaterial can also increase the compliance of the frame in a first direction, for example by reducing the cross section or surface area of a portion of the frame. The absorption of the bioabsorbable material can also allow for the controlled fracture of a portion of the frame, resulting in a sudden change in the compliance of the frame.

Other suitable frame structures can be selected from implantable frame structures disclosed in U.S. Pat. Nos. 6,730,064; 6,638,300; 6,599,275; 6,565,597; 6,530,951; 6,524,336; 6,508,833; 6,464,720; 6,447,540; 6,409,752; 6,383,216; 6,358,228; 6,336,938; 6,325,819; 6,299,604; 6,293,966; 6,200,336; 6,096,070; 6,042,606; 5,800,456; 5,755,777; 5,632,771; 5,527,354; 5,507,771; 5,507,767; 5,456,713; 5,443,498; 5,397,331; 5,387,235; 5,530,683; 5,334,210; 5,314,472; 5,314,444; 5,282,824; 5,041,126; and 5,035,706; all assigned to Cook Inc. and incorporated in their entirety herein by reference.

In other embodiments, the medical device comprises a frame having a cross section that can substantially conform to body vessel shapes that have elliptical or circular cross sections, and can change shape in response to changes in the cross section of a body vessel. Examples of such frames are provided in U.S. Provisional Patent Application 60/561,013, filed Apr. 8, 2004 by Case et al., which is incorporated herein by reference in its entirety. The expanded configuration can have any suitable cross-sectional configuration, including circular or elliptical. The expanded configuration can be characterized by a first radial compressibility along a first radial direction that is less than a second radial compressibility along a second direction.

In other embodiments, a medical device can comprise a frame and a material attached to the frame. In a preferred embodiment, the material can form one or more valve leaflets.

In some embodiments, the valve material or the support frame can comprise a remodelable material. A variety of remodelable materials are available for use in implantable medical devices. Extracellular matrix material (ECM) is one category of remodelable material. Naturally derived or synthetic collagenous materials can be used to provide remodelable surfaces on implantable medical devices. Naturally derived or synthetic collagenous material, such as extracellular matrix material, are another category of remodelable materials that include, for instance, submucosa, renal capsule membrane, dura mater, pericardium, serosa, and peritoneum or basement membrane materials. One specific example of an extracellular matrix material is small intestine submucosa (SIS). When implanted, SIS can undergo remodeling and can induce the growth of endogenous tissues upon implantation into a host. SIS has been used successfully in vascular grafts, urinary bladder and hernia repair, replacement and repair of tendons and ligaments, and dermal grafts.

The medical device can comprise extracellular matrix material derived from small intestine submocosal tissue (SIS). For example, the medical device can comprise one or more leaflets of SIS attached to a frame comprising a metallic bioabsorbable material.

SIS undergoes remodeling upon implantation into a host. SIS has been used successfully in vascular grafts, urinary bladder and hernia repair, replacement and repair of tendons and ligaments, and dermal grafts. SIS can be made, for example, in the fashion described in U.S. Pat. No. 4,902,508 to Badylak et al., U.S. Pat. No. 5,733,337 to Carr, and WIPO Patent No. WO 9822158, published May 28, 1998, issued to Cook Biotech Inc. et al. and listing Patel et al. as inventors. The preparation and use of SIS is also described in U.S. Pat. Nos. 5,281,422 and 5,275,826. Urinary bladder submucosa and its preparation is described in U.S. Pat. No. 5,554,389, the disclosure of which is expressly incorporated herein by reference. The use of submucosal tissue in sheet form and fluidized forms for inducing the formation of endogenous tissues is described and claimed in U.S. Pat. Nos. 5,281,422 and 5,275,826, the disclosures of which are expressly incorporated herein by reference.

Also provided are embodiments wherein the frame comprises a means for orienting the frame within a body lumen. For example, the frame can comprise a marker, or a delivery device comprising the frame can provide indicia relating to the orientation of the frame within the body vessel.

In some embodiments, the medical device can comprise a frame and a means for regulating fluid through a body vessel. In some embodiments, the fluid can flow through the frame, while other embodiments provide for fluid flow through a lumen defined by the frame. Some embodiments comprise a frame and a first valve member connected to the frame. A valve member, according to some embodiments, can comprise a leaflet having a free edge, responsive to the flow of fluid through the body vessel. For example, one or more valve members attached to a frame may, in one embodiment, permit fluid to flow through a body vessel in a first direction while substantially preventing fluid flow in the opposite direction. In some embodiments, the valve member comprises an extracellular matrix material, such as small intestine submucosa (SIS). The valve member can be made from any suitable material, including a remodelable material or a synthetic polymer material.

The medical devices of some embodiments can be expandable from a compressed delivery configuration to an expanded deployment configuration. Medical devices can be delivered intraluminally, for example using various types of delivery catheters, and be expanded by conventional methods such as balloon expansion or self-expansion.

Also provided are embodiments wherein the frame comprises a means for orienting the frame within a body lumen. For example, the frame can comprise a marker, or a delivery device comprising the frame can provide indicia relating to the orientation of the frame within the body vessel.

### Method Embodiments

Other embodiments provide methods of making medical devices described herein. Still other embodiments provide methods of treating a subject, which can be animal or human, comprising the step of implanting one or more support frames as described herein.

Other methods further comprise the step of implanting one or more frames attached to one or more valve members, as described herein. In some embodiments, methods of treating may also include the step of delivering a medical device to a point of treatment in a body vessel, or deploying a medical device at the point of treatment.

Methods for treating certain conditions are also provided, such as venous valve insufficiency, varicose veins, esophageal reflux, restenosis or atherosclerosis. In some embodiments, the invention relates to methods of treating venous valve-related conditions.

A “venous valve-related condition” is any condition presenting symptoms that can be diagnostically associated with improper function of one or more venous valves. In mammalian veins, venous valves are positioned along the length of the vessel in the form of leaflets disposed annularly along the inside wall of the vein which open to permit blood flow toward the heart and close to prevent back flow. These venous valves open to permit the flow of fluid in the desired direction, and close upon a change in pressure, such as a transition from systole to diastole. When blood flows through the vein, the pressure forces the valve leaflets apart as they flex in the direction of blood flow and move towards the inside wall of the vessel, creating an opening therebetween for blood flow. The leaflets, however, do not normally bend in the opposite direction and therefore return to a closed position to restrict or prevent blood flow in the opposite, i.e. retrograde, direction after the pressure is relieved. The leaflets, when functioning properly, extend radially inwardly toward one another such that the tips contact each other to block backflow of blood. Two examples of venous valve-related conditions are chronic venous insufficiency and varicose veins.

In the condition of venous valve insufficiency, the valve leaflets do not function properly. For example, the vein can be too large in relation to the leaflets so that the leaflets cannot come into adequate contact to prevent backflow (primary venous valve insufficiency), or as a result of clotting within the vein that thickens the leaflets (secondary venous valve insufficiency). Incompetent venous valves can result in symptoms such as swelling and varicose veins, causing great discomfort and pain to the patient. If left untreated, venous valve insufficiency can result in excessive retrograde venous blood flow through incompetent venous valves, which can cause venous stasis ulcers of the skin and subcutaneous tissue. Venous valve insufficiency can occur, for example, in the superficial venous system, such as the saphenous veins in the leg, or in the deep venous system, such as the femoral and popliteal veins extending along the back of the knee to the groin.

The varicose vein condition consists of dilatation and tortuosity of the superficial veins of the lower limb and resulting cosmetic impairment, pain and ulceration. Primary varicose veins are the result of primary incompetence of the venous valves of the superficial venous system. Secondary varicose veins occur as the result of deep venous hypertension which has damaged the valves of the perforating veins, as well as the deep venous valves. The initial defect in primary varicose veins often involves localized incompetence of a venous valve thus allowing reflux of blood from the deep venous system to the superficial venous system. This incompetence is traditionally thought to arise at the saphenofemoral junction but may also start at the perforators. Thus, gross saphenofemoral valvular dysfunction may be present in even mild varicose veins with competent distal veins. Even in the presence of incompetent perforation, occlusion of the saphenofemoral junction usually normalizes venous pressure.

The initial defect in secondary varicose veins is often incompetence of a venous valve secondary to hypertension in the deep venous system. Since this increased pressure is manifested in the deep and perforating veins, correction of one site of incompetence could clearly be insufficient as other sites of incompetence will be prone to develop. However, repair of the deep vein valves would correct the deep venous hypertension and could potentially correct the secondary valve failure. Apart from the initial defect, the pathophysiology is similar to that of varicose veins.

Methods for delivering a medical device as described herein to any suitable body vessel are also provided, such as a vein, artery, biliary duct, ureteral vessel, body passage or portion of the alimentary canal.

While many preferred embodiments discussed herein discuss implantation of a medical device in a vein, other embodiments provide for implantation within other body vessels. In another matter of terminology there are many types of body canals, blood vessels, ducts, tubes and other body passages, and the term “vessel” is meant to include all such passages.

The invention includes other embodiments within the scope of the claims, and variations of all embodiments, and is limited only by the claims made by the Applicants.

1. A medical device for implantation in a body vessel comprising: a support frame comprising a metallic bioabsorbable material and at least one leaflet attached to a portion of the support frame.

2. The medical device of claim 1, wherein the metallic bioabsorbable material is selected from a first group consisting of: magnesium, titanium, zirconium, niobium, tantalum, zinc, silicon and mixtures thereof.

3. The medical device of claim 1, wherein the bioabsorbable material is a bioabsorbable alloy of two or more metals.

4. The medical device of claim 3, wherein the alloy comprises a first metal selected from a first group consisting of: magnesium, titanium, zirconium, niobium, tantalum, zinc, silicon and mixtures thereof; and a second metal selected from the group consisting of: lithium, sodium, potassium, calcium, iron, manganese, and mixtures thereof.

5. The medical device of claim 3, wherein the bioabsorbable alloy is selected from the group consisting of: lithium-magnesium, sodium-magnesium, zinc-titanium and mixtures thereof.

6. The medical device of claim 3, wherein the bioabsorbable alloy further comprises gold.

7. The medical device of claim 1, where the leaflet comprises a free edge.

8. The medical device of claim 1, where the support frame defines substantially cylindrical lumen.

9. The medical device of claim 8, where the leaflet comprises a free edge that is moveable in response to the flow of fluid through the lumen.

10. The medical device of claim 8, where the leaflet permits fluid to flow through the lumen in a first direction while substantially preventing fluid flow through the lumen in the opposite direction.

11. The medical device of claim 1, comprising two or more leaflets attached to the support frame, wherein each leaflet comprises a remodelable material that is attached to one or more portions of the support frame.

12. The medical device of claim 1, wherein the medical device comprises at least two leaflets.

13. The medical device of claim 12, where the medical device comprises an opposable pair of leaflets and each of the opposable pair of leaflets comprises a flexible free edge, and where each flexible free edge of each leaflet cooperably define at least a portion of a valve orifice.

14. The medical device of claim 1, where the leaflet comprises a polyurethane material and a surface modifying agent.

15. The medical device of claim 1, where the leaflet comprises a remodelable material.

16. The medical device of claim 1, where the leaflet comprises small intestine submucosa.

17. The medical device of claim 1, where the support frame further comprises a means for orienting the support frame in a body vessel.

18. A medical device for implantation in a body vessel comprising: a support frame comprising a metallic bioabsorbable material and a means for regulating fluid in a body vessel.

19. A method of making a medical device for implantation in a body vessel, comprising the step of attaching a first valve leaflet to a support frame, the support frame comprising a metallic bioabsorbable material and at least one leaflet attached to a portion of the support frame.

20. A method of treating a subject, comprising the step of: delivering the medical device to a point of treatment in a body vessel; the medical device comprising a support frame formed at least in part from metallic bioabsorbable material and at least one leaflet attached to a portion of the support frame.
